SEQUENCE LISTING (38 sequences)

SEQ ID NO: 1 - protein, 356 aa, Bacteriophage lambda
MGRRRSHERRDLPPNLYIRNNGYYCYRDPRTGKEFGLGRDRRIAITEAIQANIELFSGHKHKPLTARINSDNSVTLHSWLDRYEKILASRGIKQKTLINYMSKIKAIRRGLPDAPLEDITTKEIAAMLNGYIDEGKAASAKLIRSTLSDAFREAIAEGHITTNHVAATRAAKSEVRRSRLTADEYLKIYQAAESSPCWLRLAMELAVVTGQRVGDLCEMKWSDIVDGYLYVEQSKTGVKIAIPTALHIDALGISMKETLDKCKEILGGETIIASTRREPLSSGTVSRYFMRARKASGLSFEGDPPTFHELRSLSARLYEKQISDKFAQHLLGHKSDTMASQYRDDRGREWDKIEIK

SEQ ID NO: 2 - DNA, 34 bp, artificial
  IntNDE-F primer
cacacatatgggaagaaggcgaagtcatgagcgc

SEQ ID NO: 3 - DNA, 46 bp, artificial
  IntECO-R primer
ctctgaattctcattatttgatttcaattttgtcccactccctgcc

SEQ ID NO: 4 - DNA, 43 bp, artificial
  attB-petF primer
ctgcttttttatactaacttggtgatgccggccacgatgcgtc

SEQ ID NO: 5 - DNA, 40 bp, artificial
  attH-petF primer
ctgctttcttataccaagtggatgccggccacgatgcgtc

SEQ ID NO: 6 - DNA, 20 bp, artificial
  aTT-petR primer
cgccacaggtgcggttgctg

SEQ ID NO: 7 - DNA, 19 bp, artificial
  Pet-F2 primer
catcggtgatgtcggcgat

SEQ ID NO: 8 - DNA, 24 bp, artificial
  Pet-RC primer
cggatatagttcctcctttcagca

SEQ ID NO: 9 - DNA, 59 bp, artificial
  pCMVattPH-QC1 primer
cattttacgtttctcgttcagctttcttatactaagttggcattataaaaaagcattgc

SEQ ID NO: 10 - DNA, 59 bp, artificial
  pCMVattPH-QC2 primer
gcaatgcttttttataatgccaacttagtataagaaagctgaacgagaaacgtaaaatg

SEQ ID NO: 11 - DNA, 20 bp, artificial
  pCMVattP-F3 primer
ccaaaaacgaggaggatttg

SEQ ID NO: 12 - DNA, 20 bp, artificial
  pCMVattP-R1 primer
actcagacaatgcgatgcaa

SEQ ID NO: 13 - DNA, 22 bp, artificial
  Int-FRev-R primer
catgacttcgccttcttcccat

SEQ ID NO: 14 - DNA, 44 bp, artificial
  IntY342A-QC1 primer
cggacaccatggcatcacaggctcgtgatgacagaggcagggag

SEQ ID NO: 15 - DNA, 44 bp, artificial
  IntY342A-QC2 primer
ctccctgcctctgtcatcacgagcctgtgatgccatggtgtccg

SEQ ID NO: 16 - DNA, 64 bp, artificial
  IntECOHIS-R primer
ctctgaattctcattagtggtgatggtgatgatgtttgatttcaattttgtcccactccctgcc

SEQ ID NO: 17 - DNA, 37 bp, artificial
  IntECO-F primer
cggaattccgatgggaagaaggcgaagtcatgagcgc

SEQ ID NO: 18 - DNA, 38 bp, artificial
  IntXBA-R primer
gctctagagctcagtgatggtgatggtgatgtaatttg

SEQ ID NO: 19 - DNA, 52 bp, artificial
  attP-PHQC1 primer
cgtttctcgttcagctttcttataccaagtgggcatattaaaaaagcattgc

SEQ ID NO: 20 - DNA, 52 bp, artificial
  attP-PHQC2 primer
gcaatgcttttttaatatgcccacttggtataagaaagctgaacgagaaacg

SEQ ID NO: 21 - DNA, 50 bp, artificial
  attB-HQC1 primer
agctagctgaagcctgctttcttataccaagtggagcgaacgcaattgaa

SEQ ID NO: 22 - DNA, 50 bp, artificial
  attB-HQC2 primer
ttcaattgcgttcgctccacttggtataagaaagcaggcttcagctagct

SEQ ID NO: 23 - DNA, 19 bp, artificial
  Rec-SYBR-F2 primer
aggtgtccactcccaggtc

SEQ ID NO: 24 - DNA, 20 bp, artificial
  Rec-SYBR-R3 primer
cgatcctctacgccggacgc

SEQ ID NO: 25 - DNA, 23 bp, artificial
  Rec-SYBR-HAT primer
ccatacgatgttccagattacgc

SEQ ID NO: 26 - DNA, 18 bp, artificial
  SS-F primer
ggcaggcttgagatctgg

SEQ ID NO: 27 - DNA, 20 bp, artificial
  SYBRgDNA-R primer
aagtcagtcccatcccagaa

SEQ ID NO: 28 - DNA, 21 bp, artificial
  attP
cagcttttttatactaagttg

SEQ ID NO: 29 - DNA, 21 bp, artificial
  attB
ctgcttttttatactaacttg

SEQ ID NO: 30 - DNA, 21 bp, artificial
  attH
ctgctttcttataccaagtgg

SEQ ID NO: 31 - DNA, 21 bp, artificial
  attPH
cagctttcttataccaagttg

SEQ ID NO: 32 - DNA, 1071 bp, artificial
  Int-h wild type integrase (with E174K mutation)
atgggaagaaggcgaagtcatgagcgccgggatttaccccctaacctttatataagaaacaatggatattactgctacagggacccaaggacgggtaaagagtttggattaggcagagacaggcgaatcgcaatcactgaagctatacaggccaacattgagttattttcaggacacaaacacaagcctctgacagcgagaatcaacagtgataattccgttacgttacattcatggcttgatcgctacgaaaaaatcctggccagcagaggaatcaagcagaagacactcataaattacatgagcaaaattaaagcaataaggaggggtctgcctgatgctccacttgaagacatcaccacaaaagaaattgcggcaatgctcaatggatacatagacgagggcaaggcggcgtcagccaagttaatcagatcaacactgagcgatgcattccgagaggcaatagctgaaggccatataacaacaaaccatgtcgctgccactcgcgcagcaaaatcaaaggtaaggagatcaagacttacggctgacgaatacctgaaaatttatcaagcagcagaatcatcaccatgttggctcagacttgcaatggaactggctgttgttaccgggcaacgagttggtgatttatgcaaaatgaagtggtctgatatcgtagatggatatctttatgtcgagcaaagcaaaacaggcgtaaaaattgccatcccaacagcattgcatattgatgctctcggaatatcaatgaaggaaacacttgataaatgcaaagagattcttggcggagaaaccataattgcatctactcgtcgcgaaccgctttcatccggcacagtatcaaggtattttatgcgcgcacgaaaagcatcaggtctttccttcgaaggggatccgcctacctttcacgagttgcgcagtttgtctgcaagactctatgagaagcagataagcgataagtttgctcaacatcttctcgggcataagtcggacaccatggcatcacagtatcgtgatgacagaggcagggagtgggacaaaattgaaatcaaataa

SEQ ID NO: 33 - DNA, 1071 bp, artificial
  Int-1 mutein of the lambda integrase
atgggaagaaggcgaagtcatgagcgccgggatttaccccctaacctttatataagaaacaatggatattactgctacagggacccaaggacgggtaaagagtttggattaggcagagacaggcgattcgcaatcactgaagctatacaggccaacattgagttattttcaggacacaaacgcaagcctctgacagcgagaatcaacagtgataattccgttacgttacattcatggcttgatcgctacgaaaaaatcctggccagcagaggaatcaagcagaagacactcataaattacatgagcaaaattaaagcaataaggaggggtctgcctgatgctccacttgaagacatcaccacaaaagaaattgcggcaatgctcaatggatacatagacgagggcaaggcggcgtcagccaagttaatcagatcaacactgagcgatgcattccgagaggcaatagctgaaggccatataacaacaaaccatgtcgctgccactcgcgcagcaaaatcaaaggtaaggagatcaagacttacggctgacgaatacctgaaaatttatcaagcagcagaatcatcaccatgttggctcagacttgcaatggaactggctgttgttaccgggcaacgagttggtgatttatgcaaaatgaagtggtctgatatcgtagatggatatctttatgtcgagcaaagcaaaacaggcgtaaaaattgccatcccaacagcattgcatattgatgctctcggaatatcaatgaaggaaacacttgataaatgcaaagagattcttggcggagaaaccataattgcatctactcgtcgcgaaccgctttcatccggcacagtatcaaggtattttatgcgcgcacgaaaagcatcaggtctttccttcgaaggggatccgcctacctttcacgagttgcgcagtttgtctgcaagactctatgagaagcagataagcgataagtttgctcaacatcttctcgggcataagtcggacaccatggcatcacagtatcgtgatgacagaggcagggagtgggacaaaattgaaatcaaataa

SEQ ID NO: 34 - DNA, 1071 bp, artificial
  Int-2 mutein of the lambda integrase
atgggaagaaggcgaagtcatgagcgccgggatttaccccctaacctttatataagaaacaatggatattactgctacagggacccaaggacgggtaaagagtttgggttaggcagagacaggcgaatcgcaatcactgaagctatacaggccaacattgagttattttcaggacacaaacacaagcctctgacagcgagaatcaacagtgataattccgttacgttacattcatggcttgatcgctacgaaaaaatcctggccagcagaggaatcaagcagaagacactcataaattacatgagcaaaattaaagcaataagggggggtctgcctgatgctccacttgaagacatcaccacaaaagaaattgcggcaatgctcaatggatacatagacgggggcaaggcggcgtcagccaagttaatcagatcaacactgagcgatgcattccgagaggtaatagctgaaggccatataacaacaaaccatgtcgctgccactcgcgcagcaaaatcaaaggtaaggagatcaagacttacgactgacgaatacctgaaaatttatcaagcagcagaatcatcaccatgttggctcagacttgcaatggaactggctgttgttaccgggcaacgagttggtgatttatgcaaaatgaagtggtctgatatcgtagatggatatctttatgtcgagcaaagcaaaacaggcgtaaaaattgccatcccaacagcattgcatattgatgctctcttattatcaatgaaggaaacacttgataaatgcaaagagattcttggcggagaaaccataattgcatctactcgtcgcgaaccgctttcatccggcacagtatcaaggtattttatgcgcgcacgaaaagcatcaggtctttccttcgaaggggatccgcctacctttcacgagttgcgcagtttgtccgcaagactctacgagaagcagataagcgataagtttgctcaacatcttctcgggcataagtcggacaccatggcatcacagtatcgtgatgacagaggcagggagtgggacaaaattgaaatcaaataa

SEQ ID NO: 35 - DNA, 1071 bp, artificial
  Int-3 mutein of the lambda integrase
atgggaagaaggcgaagtcatgagcgccgggatttaccccctaacctttatataagaaacaatggatattactgctacagggacccaaggacgggtaaagagtttggattaggcagagacaggcgaatcgcaatcactgaagctatacaggccaacattgagttattttcaggacacaaacacaagcctctgacagcgagaatcaacagtgataattccgttacgttacattcatggcttgatcgctacgaaaaaatcctggccagcagaggaatcaagcagaagacactcataaattacatgagcaaaattaaagcaataaggaggggtctgcctgatgctccacttgaagacatcaccacaagagaaattgcggcaatgctcaatggatacatagacgagggcaaggcggcgtcagccaagttaatcagatcaacactgagcgatgcattccgagaggcaatagctgaaggccatataacaacaaaccatgtcgctgccactcgcgcagcaaaatcaaaggtaaggagatcaagacttacggctgacgaatacctgaaaatttatcaagcagcagaatcatcaccatgttggctcagacttgcaatggaactggctgttgttaccgggcaacgagttggtgatttatgcaaaatgaagtggtctgatatcgtagatggatatctttatgtcgagcaaagcaaaacaggcgtaaaaattgccatcccaacagcattgcatattgatgctctcggtatatcaatgaaggaaacacttgataaacgcaaagagattcttggcggagaaaccataattgcatctactcgtcgcgaaccgctttcatccggcacagtatcaaggtattttatgcgcgcacgaaaagcatcaggtctttccttcgaaggggatccgcctacctttcacgagttgcgcagtttgtctgcaagactctacgagaagcagataagcgataagtttgctcaacatcttctcgggcataagtcggacaccatggcatcacagtatcgtgatgacagaggcagggagtgggacaaaattgaaatcaaataa

SEQ ID NO: 36 - DNA, 1071 bp, artificial
  Int-4 mutein of the lambda integrase
atgggaagaaggcgaagtcataagcgccgggatttaccccctaacctttatataagaaacaatggatattactgctacagggacccgaggacgggtaaagagtttggattaggcagagacaggcgaatcgcaatcactgaagctatacaggccaacattgagttattctcaggacacaaacgcaagcctctgacagcgagaatcaacagtgataattccgttacgttacattcatggcttgatcgctacgaaaaaatcctggccagcagaggaatcaagcagaagacactcataaattacatgagcaaaattaaagcaataaggaggggtctgcctgatgctccacttgaagacatcaccacaaaagaaattgcggcaatgctcaatggatacatagacgagggcaaggcggcgtcagccaagttaatcagatcaacactgagcgatgcattccgagaggcaatagctgaaggccatataacaacaaaccatgtcgctgccactcgcgcagcaaaatcaaaggtaaggagatcaagacttacggctgacgaatacctgaaaatttatcaagcagcagaatcatcaccatgttggctcagacttgcaatggaactggctgttgttaccgggcaacgagttggtgatttatgcaaaatgaagtggtctgatatcgtagatggatatctttatgtcgagcaaagcaaaacaggcgtaaaaattgccatcccaacagcattgcatattgatgctctcggaatatcaatgaaggaaacacttgataaatgcaaagagattcttggcggagaaaccataattgcatctactcgtcgcgaaccgctttcatccggcacagtatcaaggtattttatgcgcgcacgaaaagcatcaggtctttccttcgaaggggatccgcctacctttcacgagttgcgcagtttgtctgcaagactctatgagaagcagataagcgataagtttgctcaacatcttctcgggcataagtcggacaccatggcatcacagtatcgtgatgacagaggcagggagtgggacaaaattgaaatcaaataa

SEQ ID NO: 37 - DNA, 1071 bp, artificial
  Int-5 mutein of the lambda integrase
atgggaagaaggcgaagtcatgagcgccgggatttaccccctaacctttatataagaaacaatggatattactgctacagggacccaaggacgggtaaagagtttggattaggcagagacaggcgattcgcaatcactgaagctatacaggccaacattgagttattttcaggacacaaacacaagcctctgacagcgagaatcaacagtgataattccgttacgttacattcatggcttgatcgctacgaaaaaatcctggccagcagaggaatcaagcagaagacactcataaattacatgagcaaaattaaagcaataaggaggggtctgcctgatgccccacttgaagacatcaccacaaaagaaattgcggcaatgctcaatggatacatagacgagggcaaggcggcgtcagccaagttaatcagatcaacactgagcgatgcattccgagaggcaatagctgaaggccatataacaacaaaccatgtcgctgccactcgcgcagcaaaatcaaaggtaaggagatcaagacttacggctgacgaatacctgaaaatttatcaagcagcagaatcatcaccatgttggctcagacttgcaatggaactggctgttgttaccgggcaacgagttggtgatttatgcaaaatgaagtggtctgatatcgtagatggatatctttatgtcgagcaaagcaaaacaggcgtaaaaattgccatcccaacagcattgcatattgatgctctcggaatatcaatgaaggaaacacttgataaatgcaaagagattcttggcggagaaaccataattgcatctactcgtcgcgaaccgctttcatccggcacagtatcaaggtattttatgcgcgcacgaaaagcatcaggtctttccttcgaaggggatccgcctacctttcacgagttgcgcagtttgtctgcaagactctacgagaagcagataagcgataagtttgctcaacatcttctcgggcataagtcggacaccatggcatcacagtatcgtgatgacagaggcagggagtgggacaaaattgaaatcaaataa

SEQ ID NO: 38 - DNA, 1071 bp, artificial
  Int-6 mutein of the lambda integrase
atgggaagaaggcgaagtcatgagcgccgggatttaccccctaacctttatataagaaacaatggatattactgctacagggacccgaggacgggtaaagagtttggattaggcagagacaggcgaatcgcaatcactgaagctatacaggccaacattgagttattctcaggacacaaacgcaagcctctgacagcgagaatcaacagtgataattccgttacgttacattcatggcttgatcgctacgaaaaaatcctggccagcagaggaatcaagcagaagacactcataaattacatgagcaaaattaaagcaataaggaggggtctgcctgatgctccacttgaagacatcaccacaagagaaattgcggcaatgctcaatggatacatagacgagggcaaggcggcgtcagccaagttaatcagatcaacactgagcgatgcattccgagaggcaatagctgaaggccatataacaacaaaccatgtcgctgccactcgcgcagcaaaatcaaaggtaaggagatcaagacttacggctgacgaatacctgaaaatttatcaagcagcagaatcatcaccatgttggctcagacttgcaatggaactggctgttgttaccgggcaacgggttggtgatttatgcaaaatgaagtggtctgatatcgtagatggatatctttatgtcgagcaaagcaaaacaggcgtaaaaattgccatcccaacagcattgcatattgatgctctcggaatatcaatgaaggaaacacttgataaatgtaaagagattcttggcggagaaaccataattgcatctactcgtcgcgaaccgctttcatccggcacagtatcaaggtattttatgcgcgcacgaaaagcatcaggtctttccttcgaaggggatccgcctacctttcacgagttgcgcagtttgtctgcaagactctatgagaagcagataagcgataagtttgctcaacatcttctcgggcataagtcggacaccatggcatcacagtatcgtgatgacagaggcagggagtgggacaaaattgaaatcaaataa

(19) United States
(12) Patent Application Publication
(10) Pub. No.: US 2013/0017578 A1
(43) Pub. Date: Jan. 17, 2013

(54) MUTEINS OF THE BACTERIOPHAGE LAMBDA INTEGRASES

(71) Applicants: Farid John Ghadessy, Singapore (GB); Mei Sian Yvonne Tay, Singapore (SG); Peter Drodge, Singapore (SG)

(72) Inventors: Farid John Ghadessy, Singapore (GB); Mei Sian Yvonne Tay, Singapore (SG); Peter Drodge, Singapore (SG)

(21) Appl. No.: 13/508,352

(22) PCT Filed: Nov. 8, 2010
(86) PCT No.: PCT/SG2010/000424

§ 371 Date: Sep. 21, 2012

(30) Foreign Application Priority Data
Nov. 6, 2009 (SG) 200907415-4

(51) Int. Cl. C12N 9/12; C12N 15/87; C12P 19/34

(52) U.S. Cl. 435/91.5; 435/194; 435/462

(57) ABSTRACT

The present invention refers to muteins of the bacteriophage lambda integrases and to nucleic acid molecules comprising a nucleotide sequence encoding such muteins of the lambda integrases. The invention further refers to host cells containing a nucleic acid molecule comprising a nucleotide sequence encoding such muteins of the lambda integrases. The invention also refers to methods of recombining nucleic acids of interest into target nucleic acids in the presence of the muteins of the lambda integrases, as well as sequence specific recombination kits.

### FIELD OF THE INVENTION

This application claims the benefit of priority of Singapore patent application No. 200907415-4, filed 6 Nov. 2009, the content of which being hereby incorporated by reference in its entirety for all purposes.

The present invention refers to muteins of the bacteriophage lambda integrases and to nucleic acid molecules comprising a nucleotide sequence encoding such muteins of the lambda integrases. The invention further refers to host cells containing a nucleic acid molecule comprising a nucleotide sequence encoding such muteins of the lambda integrases. The invention also refers to methods of recombining nucleic acids of interest into target nucleic acids in the presence of the muteins of the lambda integrases, as well as sequence specific recombination kits.

### BACKGROUND OF THE INVENTION

Controlled genome modifications are well-established methods for studying the function(s) of specific genes in living organisms. The use of DNA recombinases directing the manipulation of transgenes are essential tools for controlled genome modifications. DNA recombinases catalyze the cleavage and rejoining of DNA strands between two identified nucleotides or recombination sequences. The recombinases which are generally used for the manipulation of eukaryotic genomes at present belong to the integrase family such as Cre recombinase of the bacteriophage P1 and Flp recombinase from yeast.

Notably, the Cre and Flp recombinases have been developed into powerful tools facilitating excision, integration, inversion and translocations of DNA segments between their respective recombination target sites (also referred to as cognate sites). However, the manipulation of genomes with the Cre and Flp recombinases respectively, shows significant disadvantages. In case of deletion such as the recombination of two tandem repeated loxP or FRT cognate sites in a genome, there is an irreversible loss of the DNA segment lying between the tandem repeats. Thus, a gene located on this DNA segment may be permanently lost to the cell and the organism. Therefore, the reconstruction of the original state for a new analyses of the gene function e.g. in a later developmental stage of the organism would not be possible. The irrevocable loss of the DNA segment caused by deletion may be avoided by inversion of the respective DNA segment. A gene may be inactivated by an inversion without being lost and may be switched back again at a later developmental stage or in the adult animal by means of a timely regulated expression of the recombinase via back-recombination. However, the use of both Flp and Cre recombinases in this modified method has the disadvantage that the inversion cannot be regulated as the recombination sequences will not be altered as a result of the recombination event.

Furthermore, in the case of Flp recombinases which have reduced heat stability at 37° C., the efficiency of the recombination reaction is limited in higher eukaryotes, for example in mice. Therefore, Flp recombinases have been constructed having higher heat stability than the wild type recombinases. However, these mutant Flp recombinases still show lower recombination efficiency than the Cre recombinase.

The use of both Cre and Flp recombinases can be used to integrate a desired DNA segment into the genome of a respective mammalian cell. Both Cre and Flp recombinases may catalyze intermolecular recombination. However, it would be more desirable that this reaction is feasible with “naturally” occurring cognate sites in the eukaryotic genome. Therefore, the absence of endogenous cognate sites in mammalian genomes usually requires these to be stably introduced through either homologous recombination, e.g. in mouse embryonic stem cells, or by random integration. The “primed”, pre-determined locus is then amenable to targeted manipulation by site-specific recombination reactions.

A potential strategy to overcome this limitation is to engineer recombinases with altered site specificities. To this end, Cre recombinase variants have been described that are able to specifically recombine novel target sites and excise proviral genomic DNA such as HIV in mammalian cells. Flp and bacteriophage phiC31 recombinase variants have also been described that utilize native genomic sequences as recombination target sites. Other approaches include chimeric enzymes comprising of a recombinase domain fused to zinc finger modules with defined DNA binding specificities. Site-specific zinc finger nucleases that stimulate homologous recombination at the site of an induced genomic DNA double strand break represent another strategy for achieving directed gene replacement inside eukaryotic cells.

The use of lambda integrases has been subject to extensive research for catalyzing site-specific DNA recombination. For example, two mutant lambda integrases, Int-h (E174K) and its derivative Int-h/218 (E174K/E218K) have been described and were shown to catalyze intermolecular recombination reactions at least as efficiently as the corresponding intramolecular recombination reactions in human cells. Although the presence of arm-site sequences have been shown to increase the recombination of core-sites by Int-h/218 in vivo, given the absence of an attB site in the human genome, recombination reactions occur in non-cognate sites in an essentially random manner. This makes it difficult to engineer cell lines in a controlled, reproducible fashion.

Therefore, there remains a need to engineer mutant integrases having greater efficiency and specificity in catalyzing site specific recombination reactions.

This need is solved by the muteins of the present invention and their uses.

### SUMMARY OF THE INVENTION

In one aspect, the invention provides a mutein of the lambda integrase (Swiss-Prot Accession Number PO₃₇₀₀) wherein at least one of the amino acid residues at sequence positions 6 to 41, 43 to 63, 65 to 98 and 100 to 168 of the natural amino acid sequence of the lambda integrase as set forth in SEQ ID NO: 1 is mutated.

In another aspect, the invention provides a nucleic acid molecule. The nucleic acid molecule includes a nucleotide sequence encoding a mutein as described above.

In a further aspect, the invention provides a host cell. The host cell includes a nucleic acid molecule as described above.

In yet another aspect, the invention provides a method of recombining a nucleic acid of interest into a target nucleic acid. The method includes contacting a targeting nucleic acid comprising the nucleic acid of interest with the target nucleic acid in the presence of a mutein as described above.

In yet a further aspect, the invention provides a sequence specific recombination kit. The kit includes a targeting nucleic acid into which a nucleic acid of interest can be inserted, and a mutein as described above.

The present invention is further described in the following detailed description and with reference to the following brief description of the drawings.

### DETAILED DESCRIPTION OF THE INVENTION

The present invention is based on the surprising finding that the muteins as described herein provide greater specificity and recombination efficiency between non-cognate sites of interest. Such muteins can thus be applied to enlarge the tool box for controlled genome manipulations. The muteins as described herein are also useful as reagents tools, for example when used for in vitro cloning of PCR generated products.

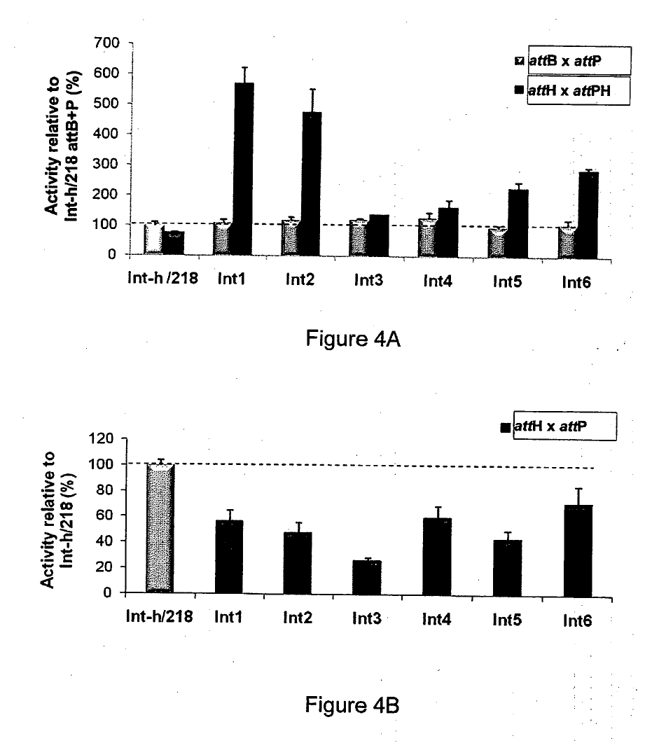

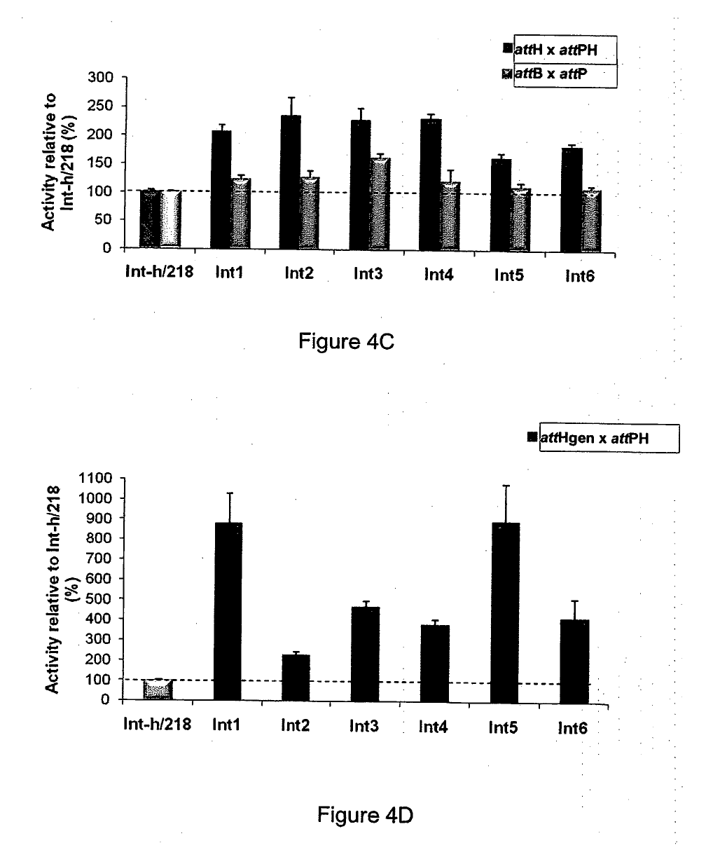

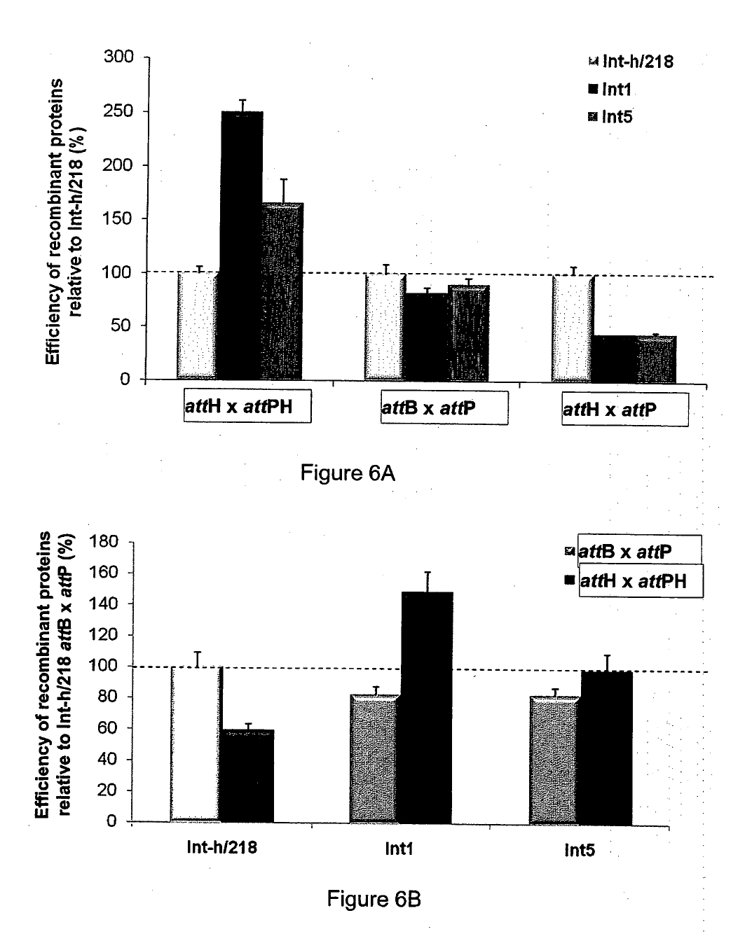

In the first aspect, there is provided a mutein of the lambda integrase (Swiss-Prot Accession Number PO₃₇₀₀) wherein at least one of the amino acid residues at sequence positions 6 to 41, 43 to 63, 65 to 98 and 100 to 168 of the natural amino acid sequence of the lambda integrase as set forth in SEQ ID NO: 1 is mutated. In this context, the muteins as described herein display significantly enhanced recombination activity on a non-cognate target DNA sequence (see FIGS. 4 and 6). As an example, the muteins displays up to about 9-fold increased recombination activity over the parental enzyme (See FIG. 4D). The parental enzyme described herein refers to an Int mutein (Int-h/218) bearing two activating mutations (E174K/E218K) in the catalytic core domain of the lambda integrase previously described.

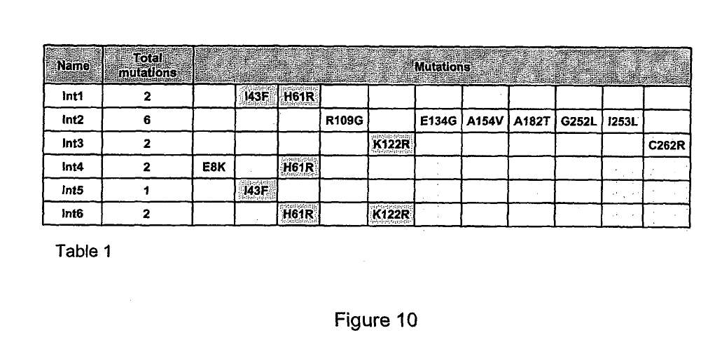

In this context, mutations present in the muteins described herein may comprise any mutations such as substitutions, deletions and also insertions of the natural amino acid sequence of the lambda integrase as long as the resulting polypeptide folds into a three-dimensionally stable structure and shows the desired (enhanced) recombination activity (see for example FIG. 10). The muteins described herein may comprise conservative and/or non-conservative mutations. Examples of possible mutations are conservatively modified variations where the alteration is the substitution of an amino acid with a chemically similar amino acid. In addition to the above, the lambda integrase may comprise mutations, such as conservative mutations, outside of the regions as mentioned above. Such conservative substitutions are known to those of skill in the art and may include substitutions between: 1) alanine, serine, threonine; 2) aspartic acid and glutamic acid; 3) asparagine and glutamine; 4) arginine and lysine; 5) isoleucine, leucine, methionine, valine; and 6) phenylalanine, tyrosine, tyroptophan.

The “amino acid residue” as used herein refers to any amino acid and can either be in the D or L form or to an amino acid mimetic that can be incorporated into a polypeptide by an amide bond. Accordingly, the positively charged amino acid residue at position 61 can for example either be a naturally occurring amino acid residue that is positively charged under physiological conditions such as arginine or lysine or a non-natural mimetic such as a lysine residue the α-amino group of which is alkylated in order to yield a (quarternary) ammonium-salt having a permanent positive charge.

In some embodiments, the mutein according to the invention comprises a mutation of at least one of the amino acid residues at sequence positions 6 to 36, 6 to 30, 6 to 24, 6 to 19, 6 to 14, 6 to 10, 10 to 41, 16 to 41, 22 to 41, 30 to 41, 35 to 41, 43 to 61, 43 to 54, 43 to 48, 46 to 63, 51 to 63, 56 to 63, 59 to 63, 65 to 94, 65 to 86, 65 to 80, 65 to 72, 65 to 69, 71 to 98, 79 to 98, 86 to 98, 92 to 98, 100 to 160, 100 to 151, 100 to 144, 100 to 137, 100 to 130, 100 to 124, 100 to 119, 100 to 110, 108 to 168, 119 to 168, 128 to 168, 136 to 168, 145 to 168 and 150 to 168 of the natural amino acid sequence of the lambda integrase as set forth in SEQ ID NO: 1.

In other embodiments, the mutein according to the invention comprises a mutation of at least one of the amino acid residues at sequence positions 43 to 61, 43 to 54, 43 to 48, 46 to 63, 51 to 63, 56 to 63, 59 to 63, 100 to 160, 100 to 151, 100 to 144, 100 to 137, 100 to 130, 100 to 124, 108 to 168 and 119 to 168 of the natural amino acid sequence of the lambda integrase as set forth in SEQ ID NO: 1.

In certain embodiments, at least one of the amino acid residues at sequence positions 43, 61 and 122 of the natural amino acid sequence of the lambda integrase as set forth in SEQ ID NO: 1 is mutated.

In some embodiments, the amino acid residue isoleucine at sequence position 43 can be replaced by an aromatic amino acid. The aromatic amino acid can be selected from any one of phenylalanine, tyrosine and tryptophan. In other embodiments, the amino acid residue isoleucine at sequence position 43 can be replaced by phenylalanine.

In some embodiments, the amino acid residue histidine at sequence position 61 can be replaced by a positively charged amino acid. The positively charged amino acid can be arginine or lysine. In other embodiments, the amino acid residue histidine at sequence position 61 can be replaced by arginine.

In some embodiments, the amino acid residue lysine at sequence position 122 can be replaced by a positively charged amino acid. The positively charged amino acid can be arginine.

Without being bound to any particular theory, it is noted that the histidine at sequence position 61 of the natural amino acid sequence of the lambda integrase resides in a loop region identified as a potential swivel about which the N-domains can rotate. Therefore, the histidine at sequence position 61 may act in concert with isoleucine at sequence position 43 of the natural amino acid sequence of the lambda integrase. This could further impact on the N-terminal DNA binding domain conformations driving allosteric control of the integrase activity.

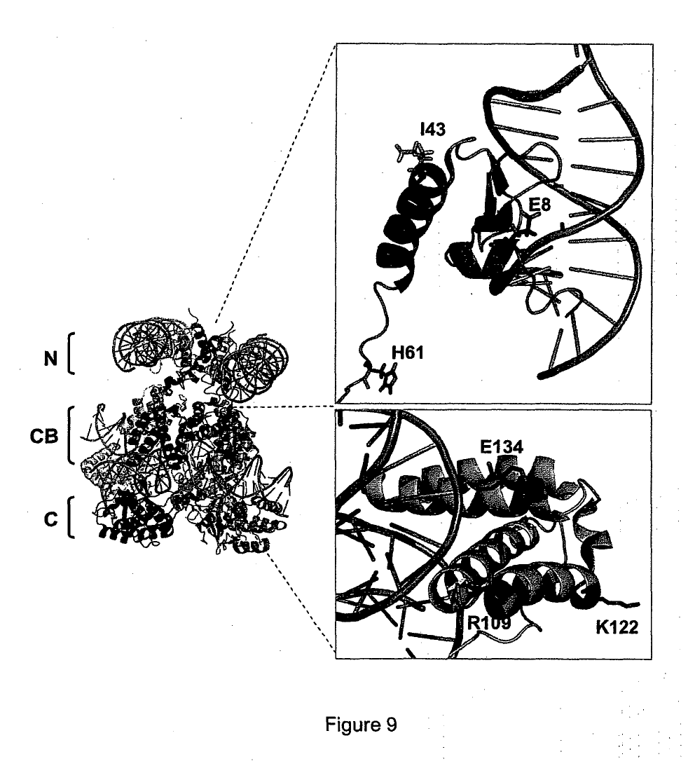

The muteins as described herein having mutations in particularly in the N-terminal domain of the lambda integrase are generally important in directing recombinase specificity and efficiency. As illustrated in FIG. 9 for example and without wishing to be bound by theory, neither I43 nor H61 of the N-terminal domain of the lambda integrase are thought to interact directly with the arm-site DNA. I43 resides on an α-helix buttressing a three-stranded anti-parallel β-sheet and flexible N-terminal tail that interact with the major and minor groove of arm site DNA, respectively. In the tetrameric structure, it is orientated away from the DNA towards the opposing helix of an adjacent Int protomer. Modeling indicates that mutation to the bulkier phenylalanine at sequence position 43 may potentially induce a steric clash with I43 of the opposing protomer's α-helix. This may influence the dynamics of the N-terminal allosteric rearrangement so as to license the recombination of non-cognate substrates. In this context, although a mutation from arginine to leucine at sequence position 42 (R42L) of a closely related bacteriophage Hong Kong 022 integrase has been described, such mutant was shown to be recombination-deficient. Therefore, mutation of at least one amino acid at the N-terminal domain for example at sequence position 43 of the lambda integrase can play a role in directing recombinase specificity and efficiency.

In other embodiments, the mutein according to the invention may comprise a further mutation of at least one of the amino acid residues at sequence positions 64 to 74 of the natural amino acid sequence of the lambda integrase as set forth in SEQ ID NO: 1. In this context, it is noted that the histidine at sequence position 61 of the natural amino acid of the lambda integrase is proximal to an α-helical coupler region (amino acid residues 64-74) that connects to the core-binding DNA domain of the lambda integrase. Mutations in the coupler region, in particularly at sequence position 64 of the natural amino acid sequence of the lambda integrase can for example, affect the directionality of recombination to favour excision over integration.

In some embodiments, the leucine residue at sequence position 64 can be replaced by a positively charged amino acid residue. In other embodiments, the positively charged amino residue can be arginine.

In certain embodiments, the mutein according to the invention comprises a mutation of at least one of the amino acid residues at sequence positions 6 to 36, 6 to 30, 6 to 24, 6 to 19, 6 to 14, 6 to 10, 43 to 61, 46 to 63, 51 to 63, 56 to 63 and 59 to 63 of the natural amino acid sequence of the lambda integrase as set forth in SEQ ID NO: 1.

In specific embodiments, at least one of the amino acid residues at sequence positions 8 and 61 of the natural amino acid sequence of the lambda integrase as set forth in SEQ ID NO: 1 is mutated.

In some embodiments, the amino acid residue glutamic acid at sequence position 8 can be replaced by a positively charged amino acid. The positively charged amino acid can be arginine or lysine. In other embodiments, the amino acid residue glutamic acid at sequence position 8 can be replaced by lysine.

In some embodiments, the mutein according to the invention may further comprise a mutation of the amino acid residue cysteine at sequence position 262 of the natural amino acid sequence of the lambda integrase as set forth in SEQ ID NO: 1. In this context, the amino acid residue cysteine at sequence position 262 can be replaced by a positively charged amino acid. The positively charged amino acid can be lysine or arginine. In some embodiments, the amino acid residue cysteine at sequence position 262 can be replaced by arginine.

In some embodiments, the mutein of the invention also comprises mutations of at least one of the amino acid residues at sequence positions 100 to 160, 100 to 151, 100 to 144, 100 to 137, 100 to 130, 100 to 124, 108 to 168, 119 to 168 and 262 of the natural amino acid sequence of the lambda integrase as set forth in SEQ ID NO: 1. In other embodiments, at least one of the amino acid residues at sequence positions 122 and 262 of the natural amino acid sequence of the lambda integrase as set forth in SEQ ID NO: 1 is mutated.

In some embodiments, the mutein of the invention may further comprise a mutation of at least one of the amino acid residues at sequence positions 182, 252 and 253 of the natural amino acid sequence of the lambda integrase as set forth in SEQ ID NO: 1.

The mutein of the invention may also comprise a mutation of at least one of the amino acid residues at sequence positions 109, 134, 154, 182, 252 and 253 of the natural amino acid sequence of the lambda integrase as set forth in SEQ ID NO: 1 (Int 2, FIG. 10).

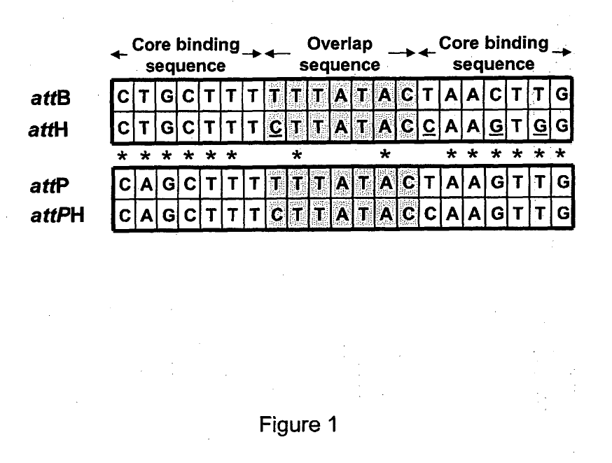

In some embodiments, the amino acid residue arginine at sequence position 109 can be replaced by glycine. In this context, the arginine residue at sequence position 109 can for example, be in contact with the phosphate backbone of the adenine residue based paired to thymidine-20 in the attB sequence (FIG. 9). Therefore, loss of this contact through mutation to glycine can influence substrate specificity to tolerate the guanidine present at this position in attH (FIG. 1).

In some embodiments, the amino acid residue glutamic acid at sequence position 134 can be replaced by glycine. In this context, the glutamic acid at sequence position 134 can for example be in the vicinity of the cytosine base-paired to the guanidine-21 in the attB sequence. It is thus found that mutation of the glutamic acid to glycine at sequence position 134 may confer acceptance of the non-canonical guanidine-20 in attH. In certain embodiments, the mutein of the invention can comprise a mutation of the amino acid residue at sequence position 109 or 134 of the natural amino acid sequence of the lambda integrase as set forth in SEQ ID NO: 1.

In some embodiments, the amino acid residue alanine at sequence position 154 can be replaced by a hydrophobic amino acid. The hydrophobic amino acid can be an aliphatic amino acid. The aliphatic amino acid can be selected from any one of isoleucine, leucine and valine. In other embodiments, the amino acid residue alanine at sequence position 154 can be replaced by valine.

In some embodiments, the amino acid residue alanine at sequence position 182 can be replaced by a hydrophilic amino acid. The hydrophilic amino acid can be a hydroxyl-containing amino acid. The hydroxyl-containing amino acid can be serine or threonine. In other embodiments, the amino acid residue alanine at sequence position 182 can be replaced by threonine.

In some embodiments, the amino acid residue glycine at sequence position 252 can be replaced by a hydrophobic amino acid. The hydrophobic amino acid can be an aliphatic amino acid. The aliphatic amino acid can be selected from any one of isoleucine, valine, alanine, and leucine. In other embodiments, the amino acid residue glycine at sequence position 252 can be replaced by leucine.

In some embodiments, the amino acid residue isoleucine at sequence position 253 can be replaced by a hydrophobic amino acid. The hydrophobic amino acid can be an aliphatic amino acid. The aliphatic amino acid can be selected from any one of valine, alanine and leucine. In other embodiments, the amino acid residue isoleucine at sequence position 253 can be replaced by leucine.

The muteins of the invention may further comprise a mutation of the amino acid residue glutamic acid at sequence position 174 of the natural amino acid sequence of the lambda integrase as set forth in SEQ ID NO: 1. In other embodiments, the muteins of the invention may further comprise a mutation of the amino acid residue glutamic acid at sequence position 218 of the natural amino acid sequence of the lambda integrase as set forth in SEQ ID NO: 1. In this context, the amino acid residue glutamic acid at sequence position 174 can be replaced by lysine. The amino acid residue glutamic acid at sequence position 218 can be replaced by lysine.

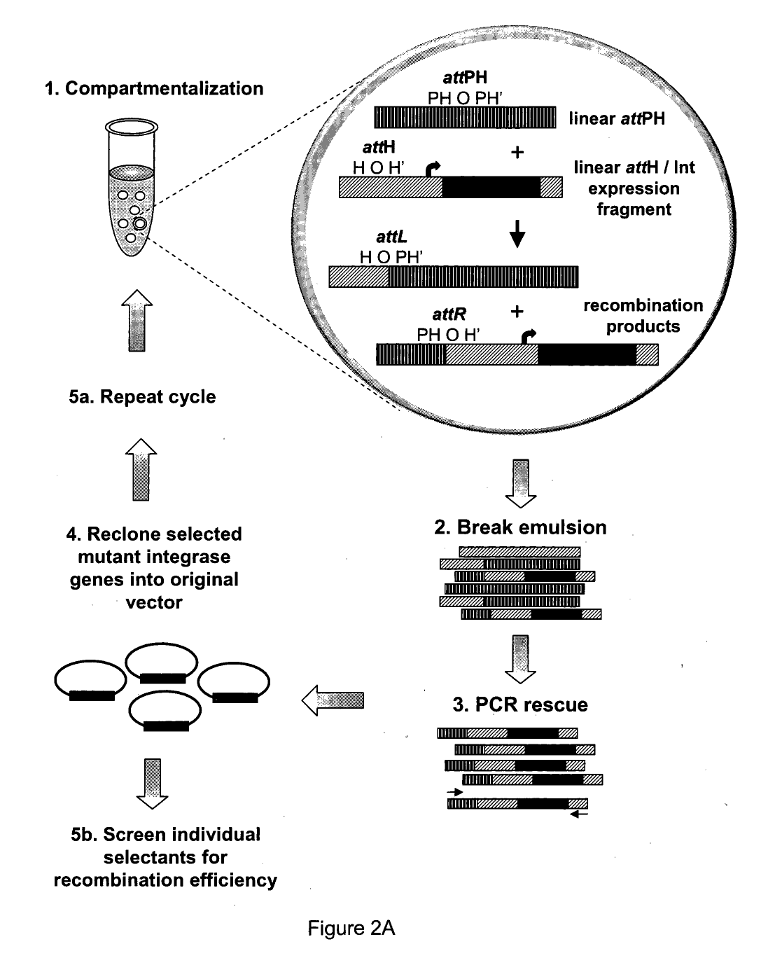

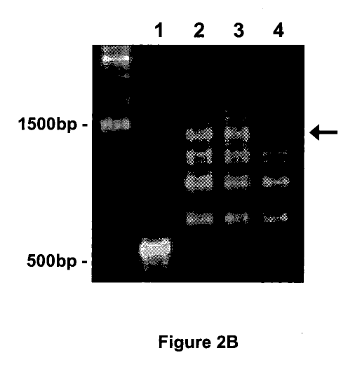

The muteins according to the invention can be generated through various selection systems known to persons skilled in the art. As an illustrative example, bacterial selection systems relying on identification of functional mutants through reporter gene activation or substrate-linked protein evolution (SLiPE) were previously described. These selection systems are one of the different methodologies for engineering altered site-specificities in recombinases. For example, a genetic selection system in yeast has also been described that yielded HIV-1 integrase variants displaying altered DNA binding affinities. As another example, in vitro compartmentalization (IVC) is used as a selection system for generating and identifying variants such as the muteins of the invention as described herein. In this context, the mutant Int-h/218 is used to construct the muteins of the invention by IVC as illustrated in FIG. 2A.

IVC is a cell-free directed evolution platform, wherein gene variants and the proteins they encode are clonally encapsulated in the aqueous compartments of an oil-in-water emulsion. IVC has been used to evolve several classes of nucleic-acid transacting proteins including methylases, transcription factors, and restriction enzymes. A related methodology utilizing compartmentalization of bacterial cells has also been used to evolve DNA polymerases with tailored properties. In vitro selection methodologies are not restricted by transformation efficiencies, and thus allow the interrogation of exceptionally large variant libraries for desired phenotypes. The use of IVC allows one to select from a considerably larger starting library (˜1.5×10¹⁰ variants) compared to bacterial-based recombinase selection systems (˜1×10⁶ variants). Therefore, IVC can be used as the selection platform which can potentially engineer other desirable features into recombinases, such as thermal stability and altered salt/pH tolerance.

As described herein, the muteins of the lambda integrase according to the invention can also be referred to as “Int” mutants, as illustrated in Table 1, FIG. 10 for example. The bacteriophage lambda integrase is the prototypical member of the large tyrosine-recombinase family. Generally, the bacteriophage lambda integrase comprises 3 distinct domains that collaborate within a higher-order tetrameric structure to form a dynamic recombinogenic complex (FIG. 9). These 3 domains are the N-terminal DNA binding domain (amino acid residues 1-64); the core-binding DNA domain (amino acid residues 65-175); and the C-terminal catalytic domain (amino acid residues 176-356). The bacteriophage lambda integrase is central to the bacteriophage lifecycle, facilitating the controlled integration and excision of its genome into and out of the host bacterial chromosome, respectively. In its natural function, the bacteriophage lambda integrase is able to catalyze site-specific recombination between a pair of target sequences, termed att sites, in the absence of high-energy cofactors. The target sequences (attP in the bacteriophage genome, attB in the bacterial genome) comprise a pair of 7 bp inverted core-binding sites separated by a 7 bp “overlap” region (FIG. 1). The “overlap region” or “overlap sequence” as used herein defines the sequence of the recombination sequences where the DNA strand exchange, including strand cleavage and religation, takes place and relates to the consensus DNA sequence 5′-TTTATAC-3′ in wild-type att sites or said sequence having functional nucleotide substitutions. The bacteriophage lambda integrase DNA core-binding domain primarily recognizes the 7 bp attP x attB core DNA sequence motifs. In the much longer attP site, the core sequence is flanked by binding sites for accessory DNA-bending factors such as integration host factor (IHF), factor for inversion stimulation (FIS) and excisionase (Xis). In addition to these accessory sites, several ‘arm’ binding sites for the N-terminal domain of the bacteriophage lambda integrase also flank the attP core site. Binding of the N-domain of the bacteriophage lambda integrase to ‘arm’ binding sites allosterically modulates the coupled core binding and catalytic domain to increase the affinity to core sites, which ultimately enables DNA strand cleavage and productive recombination of attB x attP. Therefore, these ‘arm’ regions are essential for activating efficient DNA cleavage by the C-terminal catalytic domain of bacteriophage lambda integrase, and thus contribute to the regulation of recombination directionality.

Generally, when a recombinase-mediated recombination occurs between two recognition sites, the recombination reaction can either occur on two different molecules or within the same molecule (e.g., between a recognition site on a target sequence and a recognition site on a donor sequence). In this context, the muteins according to the invention as described herein can catalyze either intermolecular or intramolecular recombination reactions or both intermolecular and intramolecular recombination reactions.

As used herein, “site-specific recombination” or “sequence-specific recombination” refers to recombination between two nucleotide sequences that each comprises at least one recognition site or at least one non-cognate site. “Site-specific” means at a particular nucleotide sequence, which can be in a specific location in the genome of a host cell for example. The nucleotide sequence can be endogenous to the host cell, either in its natural location in the host genome or at some other location in the genome, or it can be a heterologous nucleotide sequence, which has been previously inserted into the genome of the hose cell by any of a variety of known methods.

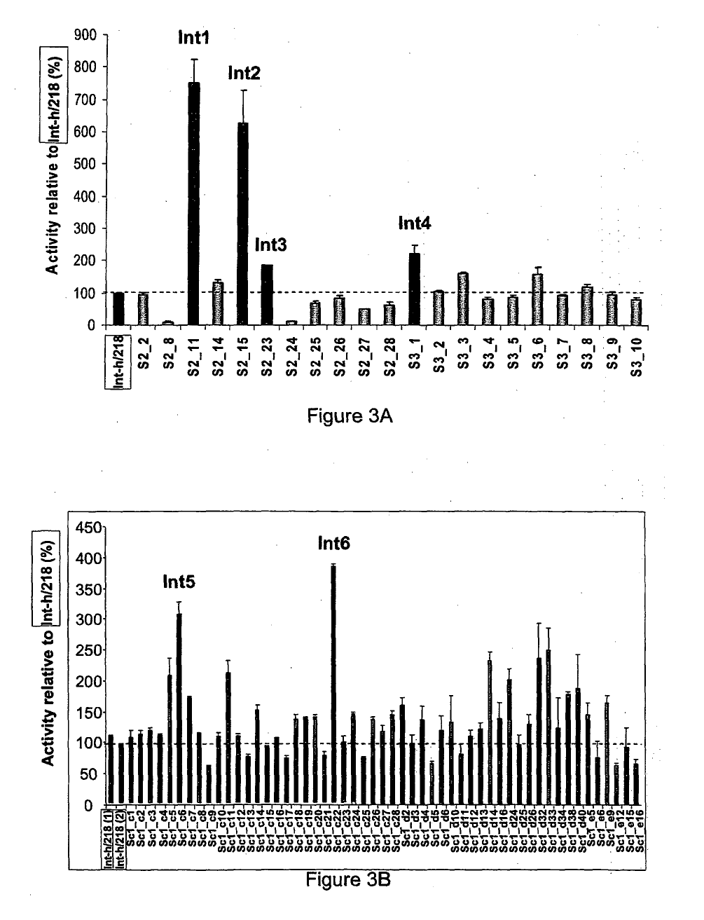

As described herein, “recognition sites” or “cognate sites” refers to a nucleotide sequence that can be recognized by a recombinase protein. The “recognition site” is the nucleotide sequence upon which binding, cleavage and strand exchange is performed by the recombinase protein and any associated accessory proteins. The lambda integrase recognizes cognate sites comprising attB, attL, attR, and/or suitable mutations of such sites. The attB site and attP sites can be recombined together by the lambda integrase, or alternatively, the attL and attR sites can be recombined by the lambda integrase. In this context, muteins (Int mutants) described herein can facilitate recombination between, for example, the attB and attP sites. The muteins described herein are able to recombine into non-cognate sites (such as the attH site) with greater efficiency, as compared to the wild type lambda integrase in its natural function (see for example FIGS. 3 and 4).

In addition, the invention is also directed to a nucleic acid molecule comprising a nucleotide sequence encoding a mutein of the invention. In certain embodiments, the nucleic acid molecule may comprise a nucleotide sequence encoding a mutein selected from any one of the muteins as exemplified in Table 1, FIG. 10. In specific embodiments, the nucleic acid molecule can comprise a nucleotide sequence as set forth in SEQ ID NO: 33, SEQ ID NO: 34, SEQ ID NO: 35, SEQ ID NO: 36, SEQ ID NO: 37 or SEQ ID NO: 38. Since the degeneracy of the genetic code permits substitutions of certain codons by other codons which specify the same amino acid and hence give rise to the same protein, the invention is not limited to a specific nucleic acid molecule but includes all nucleic acid molecules comprising a nucleotide sequence coding for the muteins of the present invention.

The nucleic acid molecule disclosed herein may comprise a nucleotide sequence encoding the mutein of the invention which can be operably linked to a regulatory sequence to allow expression of the nucleic acid molecule. A nucleic acid molecule such as DNA is regarded to be ‘capable of expressing a nucleic acid molecule or a coding nucleotide sequence’ or capable ‘to allow expression of a nucleotide sequence’ if it contains regulatory nucleotide sequences which contain transcriptional and translational information and such sequences are “operably linked” to nucleotide sequences which encode the polypeptide. An operable linkage is a linkage in which the regulatory DNA sequences and the DNA sequences sought to be expressed are connected in such a way as to permit gene sequence expression. The precise nature of the regulatory regions needed for gene sequence expression may vary from organism to organism, but shall, in general include a promoter region which, in prokaryotes, contains only the promoter or both the promoter which directs the initiation of RNA transcription as well as the DNA sequences which, when transcribed into RNA will signal the initiation of synthesis. Such regions will normally include non-coding regions which are located 5′ and 3′ to the nucleotide sequence to be expressed and which are involved with initiation of transcription and translation such as the TATA box, capping sequence and CAAT sequences. These regions can for example, also contain enhancer sequences or translated signal and leader sequences for targeting the produced polypeptide to a specific compartment of a host cell, which is used for producing a recombinant mutein of the present invention.

Accordingly, a nucleic acid of the invention can comprise a regulatory sequence, preferably a promoter sequence. In some embodiments, a nucleic acid of the invention comprises a transcriptional initiating region functional in a cell and a transcriptional terminating region functional in a cell. Suitable promoter sequences that can be used in the invention are for example, the lac promoter, the tet-promoter or the T7 promoter in the case of bacterial expression. An example of a promoter suitable for expression in eukaryotic systems is the SV 40 promoter.

In further embodiments, the nucleic acid molecule is comprised in a vector, particularly in an expression vector. Such an expression vector can comprise, besides the above-mentioned regulatory sequences and a nucleic acid sequence which codes for a mutein of the invention, a sequence coding for restriction cleavage site which adjoins the nucleic acid sequence coding for the mutein in 5′ and/or 3′ direction. This vector also allows the introduction of another nucleic acid sequence coding for a protein to be expressed or a protein part. The expression vector preferably also contains replication sites and control sequences derived from a species compatible with the host that is used for expression. The expression vector can be based on plasmids well known to person skilled in the art such as pBR322, puC16, pBluescript and the like.

The nucleic acid molecule comprising the nucleotide sequence encoding the mutein of the invention can be comprised in a vector. The vector containing the nucleic acid molecule can be transformed into host cells capable of expressing the genes. The transformation can be carried out in accordance with standard techniques. Thus, the invention is also directed to a (recombinant) host cell containing a nucleic acid molecule as defined above. In this context, the transformed host cells can be cultured under conditions suitable for expression of the nucleotide sequence encoding the mutein of the invention. Host cells can be established, adapted and completely cultivated under serum free conditions, and optionally in media which are free of any protein/peptide of animal origin. Commercially available media such as RPMI-1640 (Sigma), Dulbecco's Modified Eagle's Medium (DMEM; Sigma), Minimal Essential Medium (MEM; Sigma), CHO-S-SFMII (Invitrogen), serum free-CHO Medium (Sigma), and protein-free CHO Medium (Sigma) are exemplary appropriate nutrient solutions. Any of the media may be supplemented as necessary with a variety of compounds, examples of which are hormones and/or other growth factors (such as insulin, transferrin, epidermal growth factor, insulin like growth factor), salts (such as sodium chloride, calcium, magnesium, phosphate), buffers (such as HEPES), nucleosides (such as adenosine, thymidine), glutamine, glucose or other equivalent energy sources, antibiotics, trace elements. Any other necessary supplements may also be included at appropriate concentrations that are known to those skilled in the art.

As used herein, “nucleic acid” refers to any acid in any possible configuration, such as linearized single stranded, double stranded or a combination thereof. Nucleic acids may include, but are not limited to DNA molecules (e.g., cDNA or genomic DNA), RNA molecules (e.g., mRNA), analogues of the DNA or RNA generated using nucleotide analogues or using nucleic acid chemistry, cDNA synthetic DNA, a copolymer of DNA and RNA, oligonucleotides, and PNA (protein nucleic acids). DNA or RNA may be of genomic or synthetic origin and may be single or double stranded. A respective nucleic acid may furthermore contain non-natural nucleotide analogues and/or be linked to an affinity tag or a label.

As used herein, nucleotides include nucleoside mono-, di-, and triphosphates. Nucleotides also include modified-nucleotides, such as, but not limited to, phosphorothioate nucleotides and deazapurine nucleotides and other nucleotide analogs.

The invention also relates to a method of recombining a nucleic acid of interest into a target nucleic acid. The method includes contacting a targeting nucleic acid comprising the nucleic acid of interest with the target nucleic acid in the presence of a mutein as defined herein.

In this context, the “target nucleic acid” as used herein refers to a nucleotide sequence containing at least one recognition site. The target nucleotide sequence can be a gene, an expression cassette, a promoter, a molecular marker, a portion of any of the above, or the like. The target nucleic acid can be stably transformed into a host cell to create a transformed cell line comprising the target sequence integrated into a chromosomal location in the genome. Accordingly, in some embodiments, the target nucleic acid can include genomic DNA. The genomic DNA can be comprised in a cell. In other embodiments, the target nucleic acid can include an attH sequence (SEQ ID NO: 30).

The “targeting nucleic acid” as used herein refers to a nucleotide sequence that contains at least one recognition site. The targeting nucleic acid can contact a target nucleic acid in the presence of a mutein of the invention, in order to recombine a nucleic acid of interest into the target nucleic acid. The targeting nucleotide sequence can be a gene, an expression cassette, a promoter, a molecular marker, a portion of any of the above, or the like. In some embodiments, the targeting nucleic acid can be a vector. In other embodiments, the targeting nucleic acid comprises an attPH sequence (SEQ ID NO: 31).

The term “nucleic acid of interest” as used herein refers to a polynucleotide sequence of any length that encodes a product of interest. The selected sequence can be a full length or a truncated gene, a fusion or tagged gene, and can be a cDNA, a genomic DNA, or a DNA fragment. It can also be the native sequence, i.e., naturally occurring form(s), or can be mutated or otherwise modified as desired. These modifications can include codon optimizations to optimize codon usage in the selected cell or host cell, humanization or tagging. The selected sequence can encode a secreted, cytoplasmic, nuclear, membrane bound or cell surface polypeptide. The “product of interest” can include, but are not limited to proteins, polypeptides, fragments thereof, peptides, antisense RNA, all of which can be produced in the selected host cell.

In some embodiments, the method of recombining the nucleic acid of interest into the target nucleic acid is a sequence specific recombination. The sequence specific recombination can be performed in the presence of one or more cofactors. The cofactors can be selected from the group consisting of integration host factor (IHF), factor for inversion stimulation (FIS) and excisionase (Xis).

The method of the invention may be performed in all eukaryotic cells. Cells and cell lines may be present, for example in a cell culture and include but are not limited to eukaryotic cells, such as yeast, plant, insect or mammalian cells. For example, the cells may be oocytes, embryonic stem cells, hematopoietic stem cells or any type of differentiated cells. In certain embodiments, the method of the invention can be performed in a mammalian cell. The mammalian cell lines can include, but are not limited to a human, simian, murine, mice, rat, monkey, rabbit, rodent, hamster, goat, bovine, sheep or pig cell lines. Exemplary cell lines can include, but are not limited to Chinese hamster ovary (CHO) cells, murine myeloma cells such as NS0 and Sp2/0 cells, COS cells, Hela cells and human embryonic kidney (HEK-293) cells.

Finally, the invention also relates to a sequence specific recombination kit. The sequence specific recombination kit includes a) a targeting nucleic acid into which a nucleic acid of interest can be inserted, and b) a mutein of the invention or a nucleic acid encoding the mutein. The kit can further include a reagent for inserting the nucleic acid of interest into the targeting nucleic acid. In some embodiments, the targeting nucleic acid can include an attPH sequence. The kit can also include buffer(s) and/or instructions for recombining the nucleic acid of interest with a given target nucleic acid.

### EXEMPLARY EMBODIMENTS OF THE INVENTION

Exemplary embodiments of a mutein according to the present invention, and the methods of the invention are shown in the appended figures are described herein.

FIG. 1 shows the sequence alignment of the core bacterial attB and human attH sequences. The 7 base pairs (bp) highlighted in grey represent the overlap sequence, which must be identical in both recombination partners, i.e. attB & attP or attH & attPH. The following target recognition sequences are as follows: attP of the bacteriophage genome (SEQ ID NO: 28); attB of the bacterial genome (SEQ ID NO: 29); attH (SEQ ID NO: 30); and attPH (SEQ ID NO: 31), the attH recombination partner. The attH site differs from the bacterial attB site at one position in the 7 bp overlap sequence and three positions in the right arm core binding sequence. This attH site which is present in exon 5 of the MCT5 gene (gene accession no. SLC16A4), represents the new target recombination site (non-cognate site). The asterisk (*) below the sequences indicate nucleotides that may interact with the muteins according to the invention.

FIG. 2A shows a schematic illustration of the IVC protocol for selecting the muteins described herein, as an exemplified embodiment of the invention. (A) A schematic illustration of the IVC protocol for selecting muteins of the invention. (1) A library of muteins of the invention is constructed using error-prone PCR. Linear mutant integrase expression constructs with appended attH sites are segregated into the aqueous compartments of a water-in-oil emulsion, together with a separate linear attPH construct. Compartmentalization ensures that after translation, an active integrase mutant only recombines into the attH sequence appended to it, and not that of other integrase mutants. (2, 3, 4) The emulsion is disrupted, and genes encoding the active integrase mutants are amplified by PCR for characterization and further rounds of selection. PCR primers (arrows) only amplify recombination products containing the genes of integrase mutants capable of performing the attH x attPH recombination event.

FIG. 2B shows an example of PCR-rescue of mutein selectants generated by IVC. DNA encoding active muteins were amplified by PCR after round 1 of the selection. Lanes 1, 2: mutein library plus attPH substrate. The primer pair used in Lane 1 produces a short ˜500 bp amplicon that does not contain the full-length integrase gene, while the pair used in Lane 2 produces a longer ˜1,400 bp amplicon that contains the full-length integrase gene. Lane 3, mutein library plus attPH substrate and competing attB and attP substrates. Lane 4, negative control selection (identical to Lane 3 but with inactive IVC extract). PCR amplification in Lanes 2, 3 and 4 was performed with the same primer pair.

FIGS. 3A and 3B shows the real time PCR quantitation of recombination efficiency of the muteins of the invention obtained through an in vitro recombination assay. Integrase selectants (n=74) were subjected to a secondary screen measuring attH x attPH recombination by in vitro expressed integrase using real-time PCR. 30 muteins showed improved attH x attPH recombination compared to the parental enzyme (Int-h/218). The 6 most active muteins were selected (Int 1 to Int 6). All activities are presented relative to activity of parental Int-h/218 with the attB x attP substrates (100%). Error bars indicate standard deviation of two independent experiments.

FIG. 4A shows the improved attH x attPH recombinase activity of the 6 most active muteins (Int 1 to Int 6) translated in vitro. Out of the six muteins, both Int 1 and 2 are shown to be more efficient at performing the attH and attPH recombination reaction than the parental enzyme Int-h/218. The Int 1 and 2 are also less efficient at recombining the non-cognate attB and attP. Int 1 shows a 7-fold improved efficiency over the parental enzyme Int-h/218. All activities are presented relative to activity of parental Int-h/218 with the attB x attP substrates (100%). Error bars indicate standard deviation of two independent experiments.

FIG. 4B shows the improved recombinase activity of the 6 selected muteins (Int 1 to 6) when tested with an attH x attP substrate pair. All 6 muteins showed strongly reduced recombination efficiency (between 25-70% of Int-h/218), suggesting they have evolved to preferentially recombine attH x attPH with perfectly matched overlap sequences.

FIG. 4C shows the recombination efficiency of the 6 muteins (Int 1 to 6) tested with attH and attPH, in the presence of a 50-fold excess of wild-type attB and attP sites. Recombination of the competing attB and attP substrates within the same reaction mix was also measured. Activities are presented relative to activity of parental Int-h/218 with the attP x attPH and attB x attP substrates (100%). The 6 muteins recombined attH x attPH more efficiently than Int-h/218.

FIG. 4D shows the recombination efficiency of the 6 muteins (Int 1 to 6) tested with the attH site (endogenous) present in EcoRI-restricted human genomic DNA extracted from HEK293 cells. All six mutants recombined into the endogenous attH site (attHgen) more proficiently than Int-h/218 with Int1 and Int5 showing a 9-fold increase. Activities are presented relative to activity of parental Int-h/218 (100%).

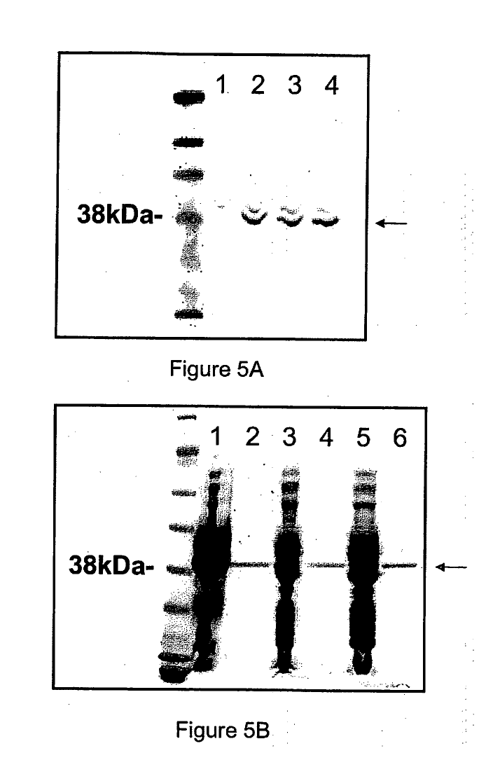

FIG. 5A shows the expression levels of the muteins of the invention by Western blot detection and analysis. Western blot detection by anti-His antibody demonstrates that the parental enzyme Int-h/218 and the muteins of the invention are expressed at similar levels in IVC extracts. Lane 1: Negative control, wild-type Int-h/218 expressed in denatured extract. Lanes 2, 3 and 4 represent Int-h/218, Int 5 and Int 1 respectively, in active extracts. Western analysis showed no difference in the in vitro expression levels of Int 1 and 5 compared to Int-h/218.

FIG. 5B shows the gel electrophoresis and Coomassie Blue staining of purified recombinant muteins expressed in E. coli. Int1 and Int5 muteins were purified and the recombination proficiencies were assayed in order to discount possible confounding effects of factors present in the in vitro expression extract. Lane 1: Int-h/218, flow-through; Lane 2: Int-h/218, purified; Lane 3: Int5, flow-through; Lane 4: Int5, purified; Lane 5: Int1, flow-through; Lane 6: Int1, purified.

FIG. 6A shows the improved recombination activity of Int 1 and Int 5 relative to Int-h/218. Real-time PCR was used to measure recombination by integrase enzymes. Recombinant mutant integrase proteins of the invention (Int1, Int5) are more efficient at performing the attH x attPH recombination reaction than recombinant parental integrase protein. They are also less efficient at recombining the non-cognate attH x attP substrate pair. Activities are presented relative to activity of Int-h/218 on each substrate pair (100%).

FIG. 6B shows the recombination activity of Int 1 and Int 5 relative to Int-h/218 attB x attP. Int 1 recombined attH x attPH 40% more efficiently than Int-h/218 recombined the attB x attP pair. Int5 processed the attH x attPH pair with the same efficiency as Int-h/218 recombined its cognate attB x attP pair. Taken together, the data show an additive effect of the I43A and H61R mutations as the double mutant (Int1) consistently outperforms the single I43A (Int5) mutant. Activities are presented relative to activity of Int-h/218 on the attB x attP substrate pair (100%). Error bars indicate standard deviation of two independent experiments.

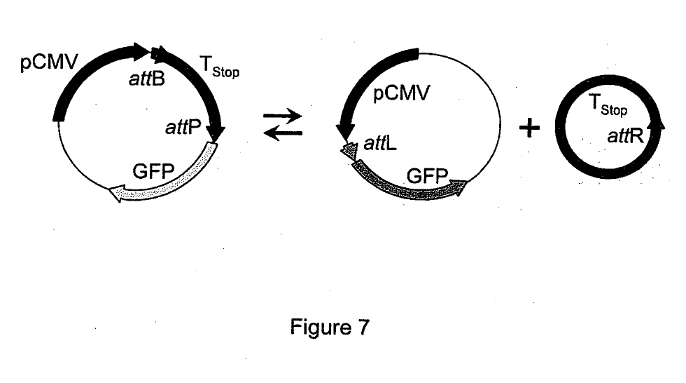

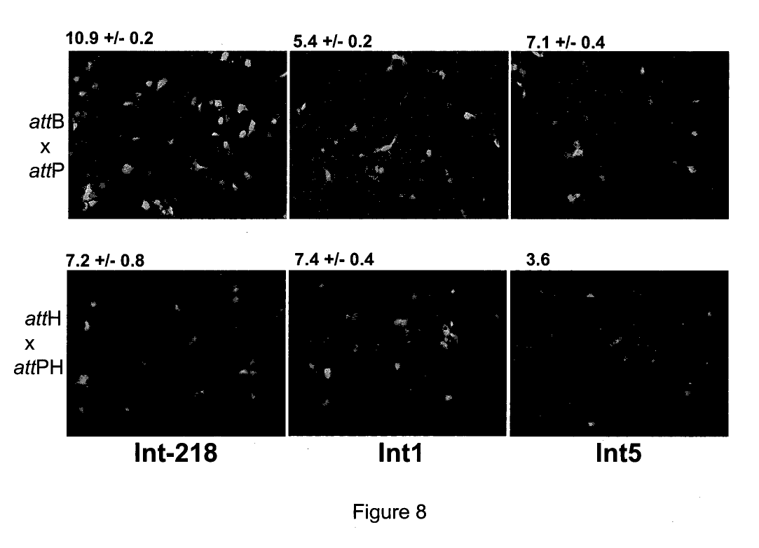

FIG. 7 shows a reporter construct containing both attB and attP sites wherein recombination of att sites flanking an inversely orientated GFP cassette results in expression of GFP. This reporter construct was used to test recombination by Int1 and Int5 in HEK293 cells (see FIG. 8).

FIG. 8 shows the preferential recombination of episomal attH x attPH substrates in HEK293 cells. Cells were transfected with GFP reporter constructs assaying attB x attP recombination (top panel) or attH x attPH recombination (lower panel) along with Int-h/218 and Int1/Int5 expression constructs. GFP expression was imaged 48 hours post-expression. Cells (n=30,000) were subsequently analysed by FACS analysis. The percentage GFP-positive cells are indicated in upper left of each pane and represents average+/−SD of two independent experiments. Int1 and Int5 processed the attB x attP substrate less efficiently than Int-h/218 (respectively 50 and 65% activity of Int-218 as measured by FACS analysis). Int1 recombined attH x attPH ˜37% more efficiently than attB x attP, again in agreement with the in vitro phenotype. Whilst Int-h/218 and Int1 showed similar efficiencies for attH x attPH as judged by numbers of GFP positive cells, the average GFP intensity for Int5 was ˜1.6 fold higher, thus indicating improved intracellular substrate processing. Int5 preferentially recombined attB x attP over attH x attPH in this assay, which is not consistent with the behavior of the recombinant purified protein measured for intermolecular recombination in vitro. In order to assay for non-specific recombination by Int1 and Int5 arising from relaxed specificity, an attH x attP GFP reporter was tested. This substrate was poorly processed by all the integrases tested (<1% activity, data not shown).

FIG. 9 shows a schematic representation of the structure of lambda integrase generated using Pymol. Left panel: quartenary structure of the lambda integrase tetramer bound to core and arm site DNA. N: N-terminal DNA binding domain; CB: core-binding DNA domain; and C: C-terminal catalytic domain. Upper right panel: Selectant mutations mapped onto the N-terminal domain. Bottom right panel: IVC-selected mutations in the CB domain that are in the vicinity of target DNA. Image created using Pymol.

FIG. 10 shows the locations and numbers of mutations present in each of the 6 most active muteins of the invention (Int 1 to Int 6) selected by IVC.

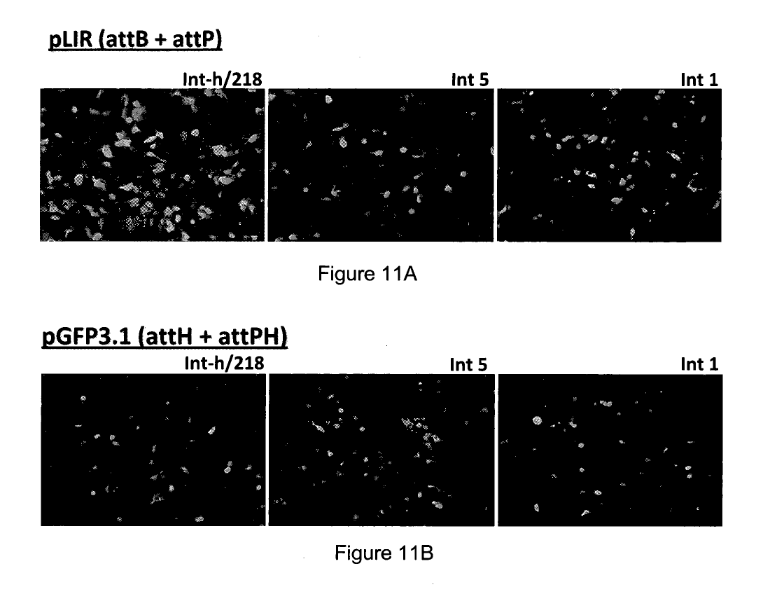

FIG. 11A shows the recombination activity of the parental and muteins (Int 1 and 5) of the invention in HEK 293 cells using attB/attP substrate pairs. The results show that the parental integrase is most proficient at recombining the endogenous attP/attB substrate as shown by highest levels of GFP expression.

FIG. 11B shows the recombination activity of the parental and muteins (Int 1 and 5) of the invention in HEK 293 cells using attH/attPH substrate pairs. Using the attH/attPH substrate integrase, the results show that Int 1 is more active than the parental integrase. Int 5 shows similar levels of activity to the parental integrase.

### EXAMPLES

The following, non limiting examples are also illustrative of the process described above and are not to be construed as limiting the scope of the present invention.

### Materials

Oligonucleotides were purchased from 1st BASE (Singapore); restriction enzymes and T4 polynucleotide kinase were from NEB; Accuzyme DNA polymerase and T7 RNA polymerase were from Bioline (UK); DNA purification kits were from Qiagen and chemical reagents were from Sigma. The following primers were used:

(SEQ ID NO: 2)
IntNDE-F:
5′-CACACATATGGGAAGAAGGCGAAGTCATGAGCGC-3′
(SEQ ID NO: 3)
IntECO-R:
5′-CTCTGAATTCTCATTATTTGATTTCAATTTTGTCCCACTCCCTGCC-3′
(SEQ ID NO: 4)
attB-petF:
5′-CTGCTTTTTTATACTAACTTGGTGATGCCGGCCACGATGCGTC-3′
(SEQ ID NO: 5)
attH-petF:
5′-CTGCTTTCTTATACCAAGTGGATGCCGGCCACGATGCGTC-3′
(SEQ ID NO: 6)
aTT-petR:
5′-CGCCACAGGTGCGGTTGCTG-3′
(SEQ ID NO: 7)
Pet-F2:
5′-CATCGGTGATGTCGGCGAT-3′
(SEQ ID NO: 8)
Pet-RC:
5′-CGGATATAGTTCCTCCTTTCAGCA-3′
(SEQ ID NO: 9)
pCMVattPH-QC1:
5′CATTTTACGTTTCTCGTTCAGCTTTCTTATACTAAGTTGGCATTATAAAAAAGCATTGC-3′
(SEQ ID NO: 10)
pCMVattPH-QC2:
5′GCAATGCTTTTTTATAATGCCAACTTAGTATAAGAAAGCTGAACGAGAAACGTAAAATG-3′
(SEQ ID NO: 11)
pCMVattP-F3:
5′-CCAAAAACGAGGAGGATTTG-3′
(SEQ ID NO: 12)
pCMVattP-R1:
5′-ACTCAGACAATGCGATGCAA-3′
(SEQ ID NO: 13)
Int-FRev-R:
5′-CATGACTTCGCCTTCTTCCCAT-3′
(SEQ ID NO: 14)
IntY342A-QC1:
5′-CGGACACCATGGCATCACAGGCTCGTGATGACAGAGGCAGGGAG-3′
(SEQ ID NO: 15)
IntY342A-QC2:
5′-CTCCCTGCCTCTGTCATCACGAGCCTGTGATGCCATGGTGTCCG-3′
(SEQ ID NO: 16)
IntECOHIS-R:
5′CTCTGAATTCTCATTAGTGGTGATGGTGATGATGTTTGATTTCAATTTTGTCCCACTCCCTGCC-3′
(SEQ ID NO: 17)
IntECO-F:
5′-CGGAATTCCGATGGGAAGAAGGCGAAGTCATGAGCGC-3′
(SEQ ID NO: 18)
IntXBA-R:
5′-GCTCTAGAGCTCAGTGATGGTGATGGTGATGTAATTTG-3′
(SEQ ID NO: 19)
attP-PHQC1:
5′-CGTTTCTCGTTCAGCTTTCTTATACCAAGTGGGCATATT AAAAAAGCATTGC-3′
(SEQ ID NO: 20)
attP-PHQC2:
5′-GCAATGCTTTTTTAATATGCCCACTTGGTATAAGAAAGC TGAACGAGAAACG-3′
(SEQ ID NO: 21)
attB-HQC1:
5′-AGCTAGCTGAAGCCTGCTTTCTTATACCAAGTGGAGCGAACGCAATTGAA-3′
(SEQ ID NO: 22)
attB-HQC2:
5′-TTCAATTGCGTTCGCTCCACTTGGTATAAGAAAGCAGGCTTCAGCTAGCT-3′
(SEQ ID NO: 23)
Rec-SYBR-F2:
5′-AGGTGTCCACTCCCAGGTC-3′
(SEQ ID NO: 24)
Rec-SYBR-R3:
5′-CGATCCTCTACGCCGGACGC-3′
(SEQ ID NO: 25)
Rec-SYBR-HAT
5′-CCATACGATGTTCCAGATTACGC-3′
(SEQ ID NO: 26)
SS-F
5′-GGCAGGCTTGAGATCTGG-3′
(SEQ ID NO: 27)
SYBRgDNA-R:
5′-AAGTCAGTCCCATCCCAGAA

### Example 1

### Vector Construction

Integrase cDNA was amplified from the vector pInt-h/218 (Christ, N. and Droge, P. (2002) Genetic manipulation of mouse embryonic stem cells by mutant lambda integrase. Genesis, 32, 203-208) using primers IntNDE-F (SEQ ID NO: 2) and IntECO-R (SEQ ID NO: 3) and ligated into the NdeI/EcoRI sites of pET-22b to generate the vector pET-Int. The 21 bp attB or attH sites were then inserted upstream of the integrase gene using primer pairs attB-petF (SED ID NO: 4)/att-petR (SEQ ID NO: 6) and attH-petF (SEQ ID NO: 5)/att-petR (SEQ ID NO: 6) respectively. Amplified products were phosphorylated using T4 polynucleotide kinase and self-ligated to generate the vectors pET-attB-Int and pET-attH-Int. A randomly mutagenized integrase cDNA library was made by error-prone PCR (38,39) using primers IntNDE-F (SEQ ID NO: 1) and IntECO-R (SEQ ID NO: 2) and the parental pInt-h/218 as a template. Sequencing of 12 random library clones indicated that 25% of the clones had a single missense mutation, 33% had two missense mutations, 17% had three or more missense mutations and 25% were wild-type. None of the subsequently selected mutations were observed. Library DNA was restricted and ligated into pET-attH vector (pET-attH-Int with the parental Int gene removed) described above. The library was next amplified by PCR using primers Pet-F2 (SEQ ID NO: 7) and Pet-RC (SEQ ID NO: 8) to generate linear DNA templates for selection. The pCMVattPH vector was constructed by site-directed mutagenesis of the parental pCMVattPmut plasmid (Christ, N., Corona, T. and Droge, P. (2002) Site-specific recombination in eukaryotic cells mediated by mutant lambda integrases: implications for synaptic complex formation and the reactivity of episomal DNA segments. J Mol Biol, 319, 305-314) using the Quikchange mutagenesis system (Stratagene) and primers pCMVattPH-QC1 (SEQ ID NO: 9) and pCMVattPH-QC2 (SEQ ID NO: 10) attPH substrate for selections was generated by PCR using primers pCMVattP-F3 (SEQ ID NO: 11) and pCMVattP-R1 (SEQ ID NO: 12) and the pCMVattPH vector template. Competitor attB and attP substrates were generated by PCR using primer pairs pet-F2 (SEQ ID NO: 7)/IntFRev-R (SEQ ID NO: 13) and pCMVattP-F3 (SEQ ID NO: 11)/pCMVattP-R1 (SEQ ID NO: 12) and plasmid templates pET-attB-Int and pCMVattPmut respectively. The parental pCMVattPmut plasmid was additionally modified to enable independent detection of attB and attH recombination events via real-time PCR (see below). This was achieved by mutating the PCR priming site Rec-SYBR-F2 (5′-AGGTGTCCACTCCCAGGTC-3′) (SEQ ID NO: 23) to Rec-SYBR-HAT (5′-CCATACGATGTTCCAGATTACGC-3′) (SEQ ID NO: 25) using mutagenic PCR. The inactive mutant Y342A integrase (41) expression construct was made by Quickchange mutagenesis of pET-attH-Int using primers IntY432A-QC1 (SEQ ID NO: 14) and IntY342A-QC2 (SEQ ID NO: 15). For purification of recombinant proteins, integrase clones were subcloned into pET-22b with a C-terminal hexahistidine tag using primers IntNDE-F (SEQ ID NO: 2) and IntECOHIS-R (SEQ ID NO: 16). For expression in HEK293 cells, integrase genes were amplified using primers IntECO-F (SEQ ID NO: 17) and IntXBA-R (SEQ ID NO: 18) and cloned in the EcoRI/XbaI sites of pcDNA3.1.

GFP-reporter vectors measuring attH x attPH and attH x attP recombination in cis were generated by Quickchange mutagenesis of the vector pXIR (32) using primer pairs attB-HQC1 (SEQ ID NO: 21)/attB-HQC2 (SEQ ID NO: 22) and attP-PHQC-1 (SEQ ID NO: 19)/attP-PHQC-2 (SEQ ID NO: 20).

### Example 2

### In Vitro Selection of Integrase Mutants

In vitro coupled transcription-translation reactions were assembled on ice in 50 μl volumes and comprised 37% (v/v) T7 extract (Novagen), 30 ng (38.1 fmol, ˜1.5×10¹⁰ integrase variants) mutant library expression template (for round 1 of selection; 5, 1, 0.5 ng used in subsequent rounds), 20 ng (50.7 fmol) attPH substrate (for round 1 of selection; 5, 1, 0.5 ng used in subsequent rounds) and 0.5 μl (2.5 units) T7 RNA polymerase. 800 ng each of competing attB and attP substrates (respectively 4.9 and 2 pmol) was also added (for rounds 1 and 2 of selection; 1200 ng was used in rounds 3 and 4). The above reaction was emulsified as previously described in Fen, C. X., Coomber, D. W., Lane, D. P. and Ghadessy, F. J. (2007) Directed evolution of p53 variants with altered DNA-binding specificities by in vitro compartmentalization. J Mol Biol, 371, 1238-1248 and then incubated at 30° C. for 45 min. The emulsion was disrupted by ether extraction and the aqueous phase purified using the DNA Clean & Concentrator™-5 Kit (Zymo Research). The purified selection products were amplified by up to 3 rounds of PCR with the sequentially nested primer pairs SS-F and PetRC, SS-F and IntECO-R, Rec-SYBR-F2 and IntECO-R, and ligated into pET-attH as described above. Expression templates for subsequent rounds of selection were then amplified via PCR using primers pET-F2 and pET-RC. After round 4 of selection, the integrase mutant library was rediversified by staggered extension process (StEP) PCR shuffling (40) of the four most active clones with the starting library.

### Example 3

### Screening of Clones

Clones were screened by in vitro coupled transcription-translation followed by real-time PCR. The individual integrase clones coupled to attH were first amplified via PCR using primers pET-F2 (SEQ ID NO: 7) and pET-RC (SEQ ID NO: 8) and then purified using the DNA Clean & Concentrator™-5 Kit as per manufacturer's instructions. Each 25 μl reaction comprised 20 ng of linear attH-integrase construct and 20 ng of linear attPH in the EcoPro™ T7 System (Novagen). Reactions were incubated for 45 min. at 37° C. Prior to real-time PCR analysis, all reactions were purified with the DNA Clean & Concentrator™-5 Kit and eluted in 12 μL water. Real-time PCR was performed with 200 nM each of primers Rec-SYBR-F2 (SEQ ID NO: 23) and Rec-SYBR-R3 (SEQ ID NO: 24) (to detect for attH x attPH recombination) and primers Rec-SYBR-HAT (SEQ ID NO: 25) and Rec-SYBR-R3 (SEQ ID NO: 24) (to detect for attB x attP recombination) using 3 μL of the eluate in a 20 μl final volume with the SYBR® GreenER™ qPCR Supermix for iCycler® (Invitrogen) on a Bio-Rad iCycler IQ™5. The cycling parameters were 95° C. for 15 seconds followed by 60° C. for 60 seconds (40 cycles). Reactions assaying for integration into endogenous attH in genomic DNA comprised 300 ng EcoRI-restricted human genomic DNA, 20 ng linear integrase construct, and 10 ng linear attPH substrate in a 25 μL in vitro transcription-translation reaction. Reactions were incubated and processed as above. The primers used for real-time quantification were Rec-SYBR-F2 (SEQ ID NO: 23) and SYBRgDNA-R (SEQ ID NO: 27).

### Example 4

### Western Blot Analysis

10 ng of linear substrate containing the parental or mutein genes was combined with the Novagen EcoPro™ T7 extract in a 20 μl reaction, and incubated for 30 min at 30° C. For Western blot analysis, 3 μl of the above reactions was diluted 5× with water and subsequently size fractionated by SDS-PAGE on 10% bis-Tris NuPAge gels in MOPS-SDS running buffer (Invitrogen) at 150 V for 90 min, and transferred to Hybond-P® PVDF membrane (GE Healthcare) in NuPage transfer buffer (Invitrogen) with 10% methanol at 40 V for 60 min at 4° C. The membrane was probed with rabbit polyclonal to the 6xhis tag (ab1187, Abcam). Antibody-protein complexes were identified by ECL-Plus (Amersham), detected on the Bio-Rad VersaDoc™ Imaging System and quantified using Quantity One 4.6.5 software.

### Example 5

### Protein Expression & Purification

2 ml cultures of E. coli BL21 (DE3) cells carrying the various pet22 integrase vectors were grown for 16 h at 37° C. in Luria-Bertani (LB) broth supplemented with Ampicillin (100 μg/ml; Sigma). 100 ml of fresh LB broth was then inoculated with 1 ml of these cultures and grown for 2-3 h to an A₆₀₀ of ˜0.5. Integrase expression was induced with 0.1 mM IPTG (Invitrogen) for 3 h; cultures were shaken at 30° C. during induction. The cells were pelleted, resuspended in binding buffer (10 mM Tris-HCl [pH 8], 150 mM NaCl, 10 mM imidazole and protease inhibitor cocktail tablet; complete mini EDTA-free, (Roche) and lysed by sonication. Cell debris was removed by centrifugation at 13,000 rpm for 45 min at 4° C. Cleared lysates containing the 6×His-tagged integrase proteins were mixed with 1.5 ml of nickel-charged resin (Ni-NTA Agarose; QIAGEN) which had been pre-equilibrated in binding buffer. The beads were subsequently rotated with the protein extracts for 3 h at 4° C. The Ni-NTA resins were then washed six times with chilled wash buffer (PBS+80 mM imidazole). Two sequential elution steps were performed, each with 120 μl of elution buffer (250 mM imidazole, 50 mM Tris-HCl [pH 8], 1 mM DTT, 150 mM NaCl and 10 mM EDTA). The amount of protein released was determined using the Quick Start™ Bradford Reagent (Bio-Rad Laboratories) as per manufacturer's instructions. The purity of all proteins was determined by Coomassie Blue staining of polyacrylamide gels (NuPAGE® Novex 4-12% Bis-Tris Gel; Invitrogen).

### Example 6

### In Vitro Recombination Assay for Purified Proteins

1 μg of purified recombinant integrase protein was incubated with 200 ng of linear attH substrate and 100 ng of linear attPH substrate for 30 min at 37° C. in recombination buffer (100 mM Tris pH 7.5, 500 mM NaCl, 25 mM DTT, 10 mM EDTA, 5 mg/ml bovine serum albumin). The reaction volume was 25 μl. 1 μl of this reaction was subsequently used for real-time PCR quantification of recombination efficiency as described above.

### Example 7

### Cell Culture & FACS Analysis

All cell culture reagents and culture plastics were obtained from Invitrogen/Gibco and Nunc, respectively, unless otherwise specified. Cell cultures were maintained at 37° C. with 5% CO₂. HEK-293 (ATCC: CRL-1573) cells were maintained in DMEM supplemented with 10% heat-inactivated FBS, 2 mM L-glutamine and 1% (v/v) penicillin/streptomycin. Co-transfection of integrase constructs in pcDNA 3.1 and pXIR reporter plasmid into 293 cells was performed in 6-well plates. 24 h before transfection, 293 cells were seeded at a density of 800,000 cells per well. 1 μg of parental or mutant integrase construct in pcDNA 3.1 was transfected per well, together with 2 μg of pXIR using Lipofectamine 2000 (Invitrogen) as per manufacturer's instructions. All transfections were carried out in duplicate. Cells were incubated for 48 hours prior to FACS analysis on the Facs Aria (Beckton Dickinson). 30,000 cells were analysed for GFP expression.

### Example 8

The present example illustrates the recombination activity of the parental and selected muteins (Int 1 and 5) of the invention in HEK 293 cells using attB/attP and attH/attPH.

All cell culture reagents and culture plastics were obtained from Invitrogen/Gibco and Nunc, respectively, unless otherwise specified. Cell cultures were maintained at 37° C. with 5% CO2. HEK-293 (ATCC: CRL-1573) cells were maintained in DMEM supplemented with 10% heat-inactivated FBS, 2 mM L-glutamine and 1% (v/v) penicillin/streptomycin. Co-transfection of Integrase constructs in pcDNA 3.1 with IRES-NEO resistance plasmids or pλIR reporter plasmid into 293 cells was performed in 6-well plates. 24 h before transfection, 293 cells were seeded at a density of 800,000 cells per well. 1 μg of the parental or the mutein construct in pcDNA 3.1 was transfected per well, together with 2 μg of pλIR or pGFP3.1 using Lipofectamine 2000 (Invitrogen) as per manufacturer's instructions. Drug selection was performed with 500 μg/ml of G418 (Sigma).

As shown in FIGS. 8 and 11, the parental integrase is most proficient at recombining the endogenous attP/attB substrate as shown by highest levels of GFP expression. However, using the attH/attPH substrate, Int 1 is more active than the parental integrase. Int 5 shows similar levels of activity to the parental integrase.

A subtle difference in substrate type may account for the weaker attH x attPH recombination phenotype displayed by Int1 and Int5 in the episomal cell-based assay when compared with the results obtained in vitro. In the cell-based assay, recombination occurs in cis, i.e. both att sites are present on the same DNA molecule. The selection strategy employed herein target sites present in trans and additionally involved substantial amounts of competitor DNA. Without being bound by any particular theory, an interesting possibility to be explored further is that the newly selected Int muteins favor intermolecular over intramolecular recombination. Another possibility for the observed discrepancy is the reduced dynamic range inherent to the GFP reporter assay compared to the real-time PCR quantitation assay.

Previous studies have shown relaxed substrate specificity to be the immediate consequence of selection pressure for altered specificity in lambda, Flp and Cre recombinases. The muteins as described herein for example, Int1 and Int5, are less proficient at recombining in vitro both attB x attP and the non-matching attH x attP substrate pair when compared to the parental Int-h/218 enzyme. Along with the latter, they are also inefficient at recombining attH x attP in the GFP reporter assay. These results suggest that the mutational drift is on a pathway towards a more restricted target site specificity. Therefore, without being bound by any particular theory, it can be anticipated that more rounds of IVC with additional selection pressure (increased competitor substrates and reduced incubation times) will yield mutants displaying further restricted specificities.

In conclusion, the muteins of the invention recombine the chosen non-cognate target sequence bearing homology to the attB sequence (termed attH) more efficiently than the parental integrase Int-h/218 recombines attB (FIGS. 4 and 6). The in vitro specificity, phenotype extended to the intracellular recombination of episomal vectors in HEK293 cells (FIG. 11). The attH sequence was identified herein bears homology to the attB sequence (FIG. 1).

Surprisingly, mutations conferring on muteins accordingly to the invention with altered specificity generally reside in the N-terminal domain of the lambda integrase. This was not previously known for directing integrase specificity. Mutations which reside in the N-terminal domain may therefore allosterically modulate integrase activity. Therefore, the muteins of the present invention provide a robust in vitro platform for the development of novel integrase muteins for biotechnological applications. Furthermore, the improved in vitro recombination by using the muteins of the present invention and the attH x attPH substrate pair indicates that the muteins described herein may be a useful reagent tool for recombination-based cloning applications.

The listing or discussion of a previously published document in this specification should not necessarily be taken as an acknowledgement that the document is part of the state of the art or is common general knowledge. All documents listed are hereby incorporated herein by reference in their entirety.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

1. A mutein of the lambda integrase wherein at least one of the amino acid residues at sequence positions 6 to 10, 43 to 48, 59 to 63, 100 to 110, 100 to 124, 100 to 137, 150 to 168 of the natural amino acid sequence of the lambda integrase as set forth in SEQ ID NO: 1 is mutated, and wherein the mutation results in altered site specificity.

2. The mutein according to claim 1, further comprising a mutation of at least one of the amino acid residues at sequence positions 182, 252, 253, and 262 of the natural amino acid sequence of the lambda integrase as set forth in SEQ ID NO: 1 is mutated.

3. The mutein according to claim 2, wherein at least one of the amino acid residues at sequence positions 8, 43, 61, 109, 122, 134, 182, 252, and 253 of the natural amino acid sequence of the lambda integrase as set forth in SEQ ID NO: 1 is substituted.

4. (canceled)

5. The mutein according to claim 1, further comprising a substitution at sequence 154 or 262 of the natural amino acid sequence of the lambda integrase as set forth in SEQ ID NO: 1.

6-43. (canceled)

44. A method of recombining a nucleic acid of interest into a target nucleic acid, the method comprising contacting a targeting nucleic acid comprising the nucleic acid of interest with the target nucleic acid in the presence of a mutein of the lambda integrase wherein at least one of the amino acid residues at sequence positions 6 to 10, 43 to 48, 59 to 63, 100 to 110, 100 to 124, 100 to 137, 150 to 168 of the natural amino acid sequence of the lambda integrase as set forth in SEQ ID NO: 1 is mutated, and wherein the mutation results in altered site specificity.

45. (canceled)

46. The method according to claim 44, wherein the target nucleic acid comprises an attH sequence.

47. (canceled)

48. The method of claim 44, wherein the targeting nucleic acid comprises an attH sequence.

49-57. (canceled)

58. The mutein according to claim 1, wherein the amino acid residue isoleucine at sequence position 43 is replaced by an aromatic amino acid.

59. The mutein according to claim 1, wherein the amino acid residue histidine at sequence position 61 is replaced by a positively charged amino acid.

60. The mutein according to claim 1, wherein the amino acid residue lysine at sequence position 122 is replaced by a positively charged amino acid.

61. The mutein according to claim 1, wherein the amino acid residue glutamic acid at sequence position 8 is replaced by a positively charged amino acid.

62. The mutein according to claim 2, wherein the amino acid residue cysteine at sequence position 262 is replaced by a positively charged amino acid.

63. The mutein according to claim 1, wherein the amino acid residue arginine at sequence position 109 is replaced by glycine.

64. The mutein according to claim 1, wherein the amino acid residue glutamic acid at sequence position 134 is replaced by glycine.

65. The mutein according to claim 1, wherein the amino acid residue alanine at sequence position 154 is replaced by a hydrophobic amino acid.

66. The mutein according to claim 2, wherein the amino acid residue alanine at sequence position 182 is replaced by a hydrophilic amino acid.

67. The mutein according to claim 2, wherein the amino acid residue glycine at sequence position 252 is replaced by a hydrophobic amino acid.

68. The mutein according to claim 2, wherein the amino acid residue isoleucine at sequence position 253 is replaced by a hydrophobic amino acid.

69. The mutein according to claim 1, further comprising a mutation of the amino acid residue glutamic acid at sequence position 174 of the natural amino acid sequence of the lambda integrase as set forth in SEQ ID NO: 1.

70. The mutein according to claim 1, further comprising a mutation of at least one of the amino acid residues at sequence positions 64 to 74 of the natural amino acid sequence of the lambda integrase as set forth in SEQ ID NO: 1.
